(19) United States
(12) Patent Application Publication
(10) Pub. No.: US 2009/0062790 A1
(43) Pub. Date: Mar. 5, 2009

(54) DIRECT VISUALIZATION BIPOLAR ABLATION SYSTEMS

(75) Inventors: Zachary J. MALCHANO, San Francisco, CA (US); Ruey-Feng PEH, Mountain View, CA (US); David MILLER, Cupertino, CA (US); Edmund TAM, Mountain View, CA (US); Vahid SAADAT, Atherton, CA (US); Aseem K. THAKUR, Sunnyvale, CA (US)

Correspondence Address: LEVINE BAGADE HAN LLP, 2483 EAST BAYSHORE ROAD, SUITE 100, PALO ALTO, CA, 94303, US

(73) Assignee: Voyage Medical, Inc., Campbell, CA (US)

(21) Appl. No.: 12/201,811

(22) Filed: Aug. 29, 2008

Related U.S. Application Data
(60) Provisional application No. 60/969,511, filed on Aug. 31, 2007.

(51) Int. Cl. A61B 18/14

(52) U.S. Cl. 606/41

(57) ABSTRACT

Direct visualization bipolar ablation systems are described which utilize bipolar electrode arrangements. Such assemblies are configured to facilitate the application of bipolar energy delivery, such as RF ablation, to an underlying target tissue for treatment in a controlled manner while directly visualizing the tissue during the bipolar ablation process.

### CROSS-REFERENCE TO RELATED APPLICATIONS

This application claims the benefit of priority to U.S. Prov. Pat. App. 60/969,511 filed Aug. 31, 2007, which is incorporated herein by reference in its entirety.

### FIELD OF THE INVENTION

The present invention relates generally to medical devices used for accessing, visualizing, and/or treating regions of tissue within a body. More particularly, the present invention relates to methods and apparatus for the delivery of ablation energy, such as radio-frequency (RF) ablation, to an underlying target tissue utilizing a bipolar electrode configuration for treatment in a controlled manner, while directly visualizing the tissue.

### BACKGROUND OF THE INVENTION

Conventional devices for visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surrounding the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging. Additionally, imaging balloons are subject to producing poor or blurred tissue images if the balloon is not firmly pressed against the tissue surface because of intervening blood between the balloon and tissue.

Accordingly, these types of imaging modalities are generally unable to provide desirable images useful for sufficient diagnosis and therapy of the endoluminal structure, due in part to factors such as dynamic forces generated by the natural movement of the heart. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens such as the interior chambers of the heart. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Thus, a tissue imaging system which is able to provide real-time in vivo images of tissue regions within body lumens such as the heart through opaque media such as blood and which also provide instruments for therapeutic procedures upon the visualized tissue are desirable.

### SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or along the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood, the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

More particularly in certain variations, the tissue visualization system may comprise components including the imaging hood, where the hood may further include a membrane having a main aperture and additional optional openings disposed over the distal end of the hood. An introducer sheath or the deployment catheter upon which the imaging hood is disposed may further comprise a steerable segment made of multiple adjacent links which are pivotably connected to one another and which may be articulated within a single plane or multiple planes. The deployment catheter itself may be comprised of a multiple lumen extrusion, such as a four-lumen catheter extrusion, which is reinforced with braided stainless steel fibers to provide structural support. The proximal end of the catheter may be coupled to a handle for manipulation and articulation of the system.

To provide visualization, an imaging element such as a fiberscope or electronic imager such as a solid state camera, e.g., CCD or CMOS, may be mounted, e.g., on a shape memory wire, and positioned within or along the hood interior. A fluid reservoir and/or pump (e.g., syringe, pressurized intravenous bag, etc.) may be fluidly coupled to the proximal end of the catheter to hold the translucent fluid such as saline or contrast medium as well as for providing the pressure to inject the fluid into the imaging hood.

In treating tissue regions which are directly visualized, as described above, treatments utilizing electrical energy may be employed to ablate the underlying visualized tissue. Many ablative systems typically employ electrodes arranged in a monopolar configuration where a single electrode is positioned proximate to or directly against the tissue to be treated within the patient body and a return electrode is located external to the patient body. Utilization of bipolar electrode ablation removes the need for a return or grounding electrode to be adhered to the skin of the patient and may further allow for a more precise delivery of ablation energy over a small target area for creation of precise lesions.

In particular, such assemblies, apparatus, and methods may be utilized for treatment of various conditions, e.g., arrhythmias, through ablation under direct visualization. Variations of the tissue imaging and manipulation apparatus may be configured to facilitate the application of bipolar energy delivery, such as radio-frequency (RF) ablation, to an underlying target tissue for treatment in a controlled manner while directly visualizing the tissue during the bipolar ablation process as well as confirming (visually and otherwise) appropriate treatment thereafter.

Various configurations may be utilized for a bipolar electrode arrangement which allows for bipolar ablation of tissue within the visual field being imaged via an imaging element. The current may be conducted between the electrodes through the transparent saline fluid infused into and through the hood. One example may include a first electrode positioned within or along the imaging hood and a second electrode positioned along the distal membrane of hood. The electrode along the hood membrane may be in a number of different configurations such as a ring electrode. Alternatively, two or more electrodes may be positioned in various arrangements over the membrane.

In other variations, the hood (or balloon in other variations) may be internally segmented into two or more separated chambers where saline fluid having opposite charges may be introduced into each respective chamber for bipolar ablation. Each chamber may define a corresponding first and second aperture over the distal membrane and may also each have a corresponding first and second electrode positioned within each respective chamber. Each electrode may be positioned within the chambers via respective first and second electrode support members. The transparent fluid may be introduced into each chamber past the electrodes such that the charged fluid passing through their respective apertures may contact one another over the tissue to conduct energy therebetween and ablate the underlying tissue. In another variation, rather than utilizing two separate chambers, a second inner hood may be positioned within the visualization hood to achieve the same or similar electrode arrangement.

In yet other variations, one or more of the support struts may be configured as electrodes well. The current may flow between the respective support struts or between a first electrode and one or more of the support struts. In yet additional variations, a bipolar electrode arrangement may be positioned along the hood and/or hood membrane such that tissue drawn into the hood or portions thereof may be ablated accordingly. In drawing portions of tissue relative to the hood, various instruments, such as tissue graspers, may also be utilized and optionally configured as an electrode as well.

Other variations may also include one or more struts having conductive tips which are configured to extend distally and project past the hood. In use, as the underlying tissue is visualized, as previously described, the one or more conducting tips may be extended distally into the tissue region surrounding the hood and contacted against the tissue surface and the conducting fluid may be infused into hood and into the area immediately surrounding the hood. The ablation energy may be thus conducted between a first electrode and the one or more conducting tips to ablate the tissue therebetween.

Additional instruments such as needles or needle assemblies may be advanced into the underlying tissue being visualized. The one or more needles may be configured as electrodes as well to allow for conduction into the underlying tissue for creating transmural lesions. Aside from needles, other instruments such as expandable anchors or ablation probe members may alternatively be utilized.

In yet other variations, a return electrode may be positioned proximally of the hood, e.g., along the deployment catheter or outer sheath. In such an arrangement, the return electrode may be positioned along a first tissue region, such as an atrial septum, while the first electrode is advanced distally such as in a left atrium of the heart. Conduction between the electrodes may thus be effected to ablate the tissue underlying and/or surrounding the electrode arrangement. In other variations, a separate instrument incorporating a return electrode may be advanced within the patient body, e.g., intravascularly or through a body cavity, and positioned in proximity to the electrode to effect ablation of the tissue region surrounding or in proximity to the electrodes.

In a further variation, ablation energy may be controlled utilizing parameters such as the salinity concentration of saline or by controlling the temperature of the transparent saline fluid, which is also utilized for visualization.

### BRIEF DESCRIPTION OF THE DRAWINGS

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIG. 6 shows an assembly view of a visualization system configured for tissue ablation utilizing a bipolar electrode configuration.

FIG. 7 shows a side view of a visualization hood configured for tissue ablation utilizing a bipolar electrode configuration with a central electrode and a ring electrode.

FIG. 8 shows a side view of another variation where a transparent fluid may conduct the charge to or from a ring electrode.

FIG. 9 shows a side view of another variation where a transparent fluid may conduct the charge to or from a central electrode.

FIG. 10 shows a side view of another variation of a visualization hood having at least two chambers through which transparent fluid conducting a current may flow to effect tissue ablation.

FIG. 11 shows a side view of another variation where a current may be flowed through the transparent fluid flowing between co-axially overlapping hoods.

FIG. 12 shows a side view of another variation where an inflatable visualization balloon may have at least two chambers through which transparent fluid conducting a current may flow to effect tissue ablation.

FIG. 13A shows a side view of another variation where a plurality of electrodes configured in a bipolar arrangement may be positioned along a distal membrane of the hood.

FIGS. 13B and 13C illustrate end views of electrode arrangement variations along the distal membrane.

FIG. 13D illustrates an end view of a concentric ring electrodes configured in a bipolar electrode arrangement.

FIG. 14A shows a side view of another variation where one or more support struts serve as electrodes.

FIG. 14B shows a side view of the variation of FIG. 14A ablating tissue via the one or more support struts.

FIG. 14C shows a side view of a visualization hood ablating tissue via energy conducted through the transparent fluid between an electrode within the hood and one or more support struts serving as electrodes.

FIG. 15 shows a side view of another variation where tissue drawn partially into the hood through the aperture is ablated between electrodes.

FIG. 16 shows a side view of another variation where tissue drawn between at least two separate chambers may be ablated as current is conducted therebetween.

FIG. 17 shows a side view of another variation where tissue drawn between two separate chambers via a tissue grasper may be ablated therebetween.

FIG. 18 shows a side view of another variation where tissue adhered to a tissue grasping instrument may be ablated through the instrument.

FIG. 19 shows a side view of another variation where a tissue grasper having at least two grasping members may ablate the tissue.

FIG. 20 shows a side view of another variation where one or more distally projecting struts may be employed as electrodes.

FIG. 21 shows a side view of another variation where one or more distally projecting conducting wires may be employed as electrodes.

FIG. 22 shows a side view of another variation where one or more distally extendable members may be employed as electrodes.

FIG. 23 shows a side view of another variation where a distally projecting needle may be employed as an electrode for tissue ablation.

FIG. 24 shows a side view of another variation where a distally projecting needle may be employed with a ring electrode for tissue ablation.

FIG. 25 shows a side view and a detail view of a multiple needle assembly having an arrangement of electrodes along the needle body.

FIG. 26 shows a side view of a another variation where a distally protruding anchor member may be utilized as an electrode for tissue ablation.

FIGS. 27A and 27B show side and end views, respectively, of a rotatable member utilized as an electrode for bipolar ablation.

FIG. 28 shows a side view of another variation where hyposaline or chilled saline may be used to reduce or control the conductivity of the fluid for tissue ablation.

FIGS. 29A and 29B show side views of a return electrode positioned optionally along the deployment catheter shaft and the sheath, respectively.

FIG. 30 illustrates a visualization catheter positioned transseptally within a left atrium where a return electrode is positioned along the sheath proximate to the atrial septum.

FIG. 31 illustrates a visualization catheter where a return electrode is positioned external to the atrial chamber along an epicardial surface.

FIG. 32 illustrates a visualization catheter where a return electrode may be configured as a basket deployed in proximity to the hood.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transseptal access to the left atrium, cannulating the coronary sinus, diagnosis of valve regurgitation/stenosis, valvuloplasty, atrial appendage closure, arrhythmogenic focus ablation, among other procedures.

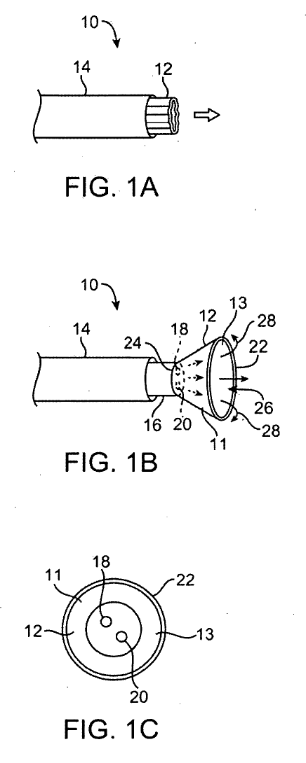

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transseptal procedure or septostomy. For procedures such as percutaneous valve repair and replacement, transseptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and outer surface 11.

Imaging hood 12 may be attached at interface 24 to a deployment catheter 16 which may be translated independently of deployment catheter or sheath 14. Attachment of interface 24 may be accomplished through any number of conventional methods.

Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

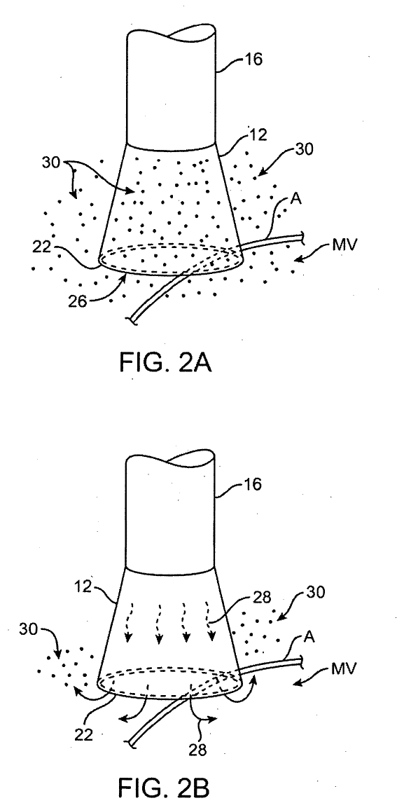

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

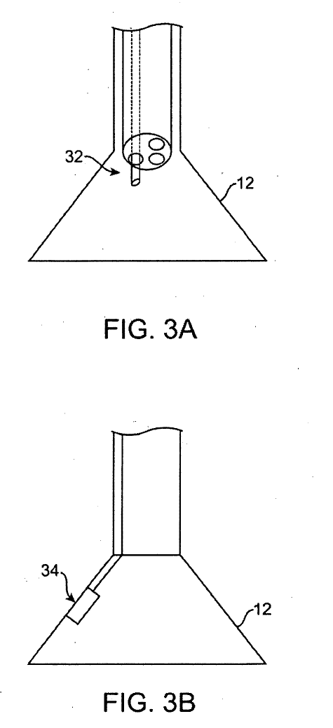

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

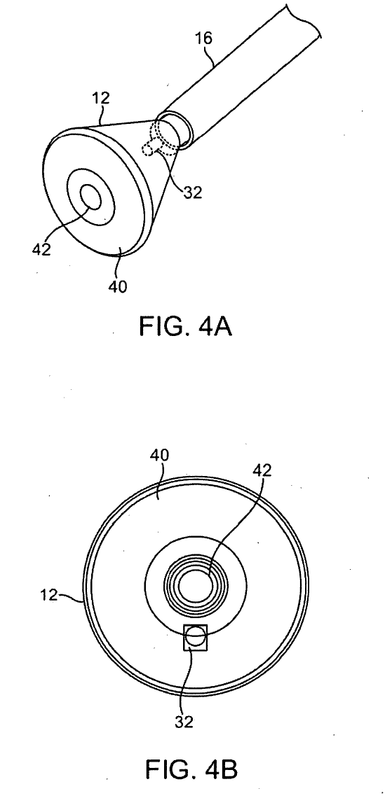

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from the hood 12 when the interior of the hood 12 is infused with the clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

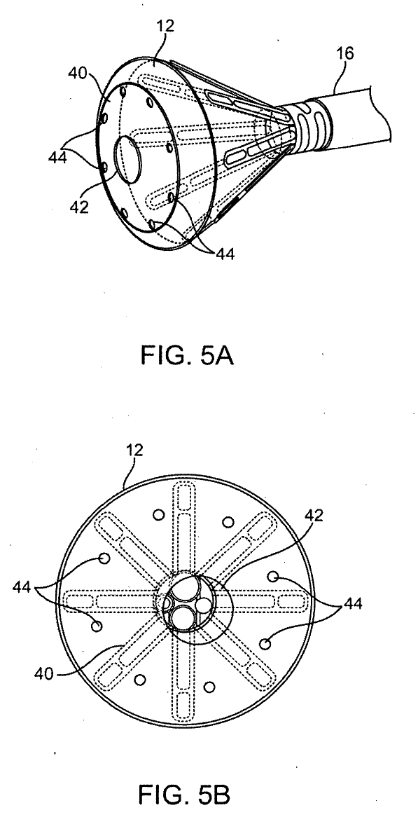

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imaging and manipulation systems and methods which may be utilized with apparatus and methods described herein are further described, for example, in U.S. patent application Ser. No. 11/259,498 filed Oct. 25, 2005 (U.S. Pat. Pub. No. 2006/0184048 A1); 11/763,399 filed Jun. 14, 2007 (U.S. Pat. Pub. No. 2007/0293724 A1); and also in 11/828,267 filed Jul. 25, 2007 (U.S. Pat. Pub. No. 2008/0033290 A1), and 11/775,837 filed Jul. 10, 2007 (U.S. Pat. Pub. No. 2008/0009747 A1) each of which is incorporated herein by reference in its entirety.

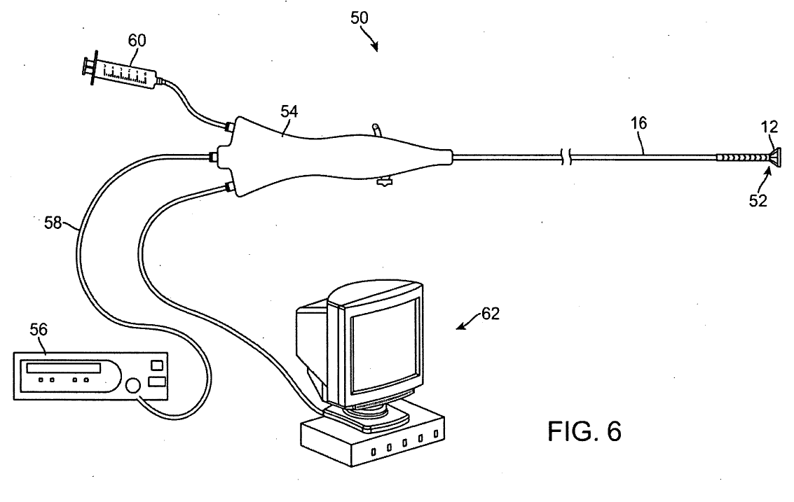

In treating tissue regions which are directly visualized, as described above, treatments utilizing electrical energy may be employed to ablate the underlying visualized tissue. Many ablative systems typically employ electrodes arranged in a monopolar configuration where a single electrode is positioned proximate to or directly against the tissue to be treated within the patient body and a return electrode is located external to the patient body. The assembly illustrated in FIG. 6 shows an example of a tissue visualization system which is configured with electrodes arranged in a bipolar configuration where the electrode and return electrode are arranged in proximity to the tissue region to be treated and visualized. Utilization of bipolar electrode ablation removes the need for a return or grounding electrode to be adhered to the skin of the patient and may further allow for a more precise delivery of ablation energy over a small target area for creation of precise lesions.

In particular, such assemblies, apparatus, and methods may be utilized for treatment of various conditions, e.g., arrhythmias, through ablation under direct visualization. Details of examples for the treatment of arrhythmias under direct visualization which may be utilized with apparatus and methods described herein are described, for example, in U.S. patent application Ser. No. 11/775,819 filed Jul. 10, 2007 (U.S. Pat. Pub. No. 2008/0015569 A1), which is incorporated herein by reference in its entirety. Variations of the tissue imaging and manipulation apparatus may be configured to facilitate the application of bipolar energy delivery, such as radio-frequency (RF) ablation, to an underlying target tissue for treatment in a controlled manner while directly visualizing the tissue during the bipolar ablation process as well as confirming (visually and otherwise) appropriate treatment thereafter.

As shown, bipolar ablation and visualization catheter assembly 50 illustrates one variation where the visualization hood 12 may incorporate a bipolar ablation assembly 52 within and/or along the hood 12. The assembly 50 is further illustrated where bipolar ablation assembly 52 may be coupled or otherwise in electrical communication with power generator 56 (e.g., RF power generator) through deployment catheter 16 and handle 54 via cable 58. Fluid reservoir 60 is also illustrated as being coupled to handle 54 and in fluid communication with hood 12 as well as image display assembly 62 which may be coupled to an optical fiber bundle or to an electronic imaging sensor (e.g., CCD or CMOS imager) positioned within or along hood 12 for visualizing the underlying tissue, as described above.

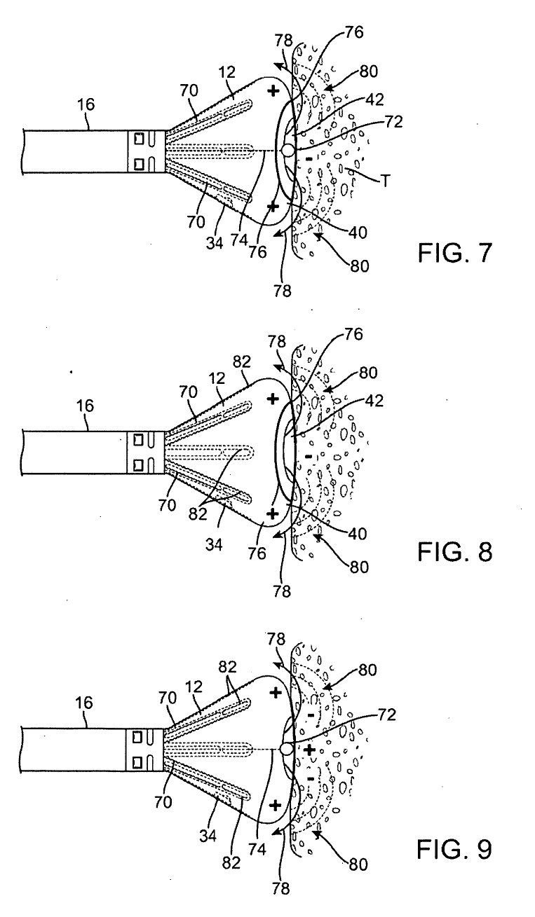

Bipolar ablation assembly 52 may be configured in a number of different arrangements to effect bipolar ablation of the underlying tissue. One example is shown in the side view of FIG. 7, which illustrates hood 12 having a number of longitudinally oriented support struts 70 therealong and membrane 40 defining aperture 42 over a distal end of hood 12. Imaging element 34 may be positioned along an inner surface of hood 12 to provide imaging the underlying tissue. In this variation, an electrode 72 may extend from catheter 16 via electrode support member 74 and positioned within hood 12, e.g., positioned near or along a central axis of aperture 42 and proximate to or through aperture 42. An electrically conductive electrode ring 76 may be positioned along the membrane 40 and enclose aperture 42. The electrically conductive central 72 and ring electrode 76 may be made from a number of bio-inert materials such as, though not limited to, stainless stain, silver, gold, platinum, etc. Moreover, electrodes 72, 76 may be connected by conductive wires which are insulated by a thin layer of insulation such as PET, latex or other biocompatible polymers.

In use, the visualization hood 12 is placed against or adjacent to a region of tissue T to be imaged and/or ablated in a body lumen that is normally filled with opaque bodily fluids such as blood. Translucent or transparent fluids 78 which are also electrically conductive, such as saline, may be then introduced into the imaging hood 12 until the transparent fluid 78 displaces the blood thus leaving a clear region of tissue T to be imaged via the imaging element 34 before an ablation process. Upon attaining visual confirmation of the target tissue T surface, RF energy may be generated from power generator 56 such that ablation energy 80 is conducted between central electrode 72 and ring electrode 76 via the saline fluid 78 flowing therebetween in effect ablating the underlying tissue. The saline fluid 78 purged from hood 12 and out through aperture 42 may thus serve multiple functions of clearing blood for visualization, conducting ablative energy, as well as optionally cooling the ablated tissue region to prevent tissue charring, desiccation, or other endothelial disruptions such as “tissue popping”. Other examples of utilizing energy conductive fluid for tissue visualization and ablation are described in further detail in U.S. patent application Ser. No. 12/118,439 filed May 9, 2008 as well as U.S. Prov Pat. App. No. 60/917,487 filed May 11, 2007, each of which is incorporated herein by reference in its entirety.

Another variation is illustrated in the side view of FIG. 8, which shows hood 12 having ring electrode 76 positioned on membrane 40 and serving as the return electrode. In this variation, RF energy may be conducted through saline 78 between one or more electrically charged support struts 70, which may have one or more portions 82 exposed along an inner surface of hood 12 in contact with the introduced saline 78, and ring electrode 76. As the saline fluid 78 is introduced into and through hood 12 and aperture 42, the ablation energy is conducted through the saline fluid 78 from the exposed electrode portions 82 and to the return ring electrode 76.

in another variation, FIG. 9 shows an example where a centrally positioned electrode 72 may be placed near or at aperture 42, as described above, and instead of utilizing a ring electrode, energy may be conducted through the saline fluid 78 between the exposed portions 82 of electrically charged support struts 70 and electrode 72 to ablate the underlying tissue. In this and other variations described herein, electrode 72 may be comprised at least in part by a transparent casing, e.g., polycarbonate polymers.

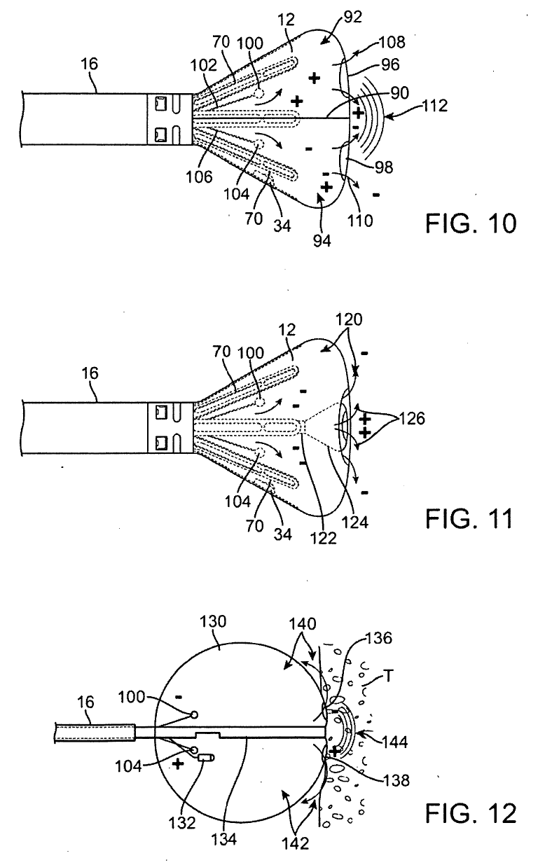

FIG. 10 illustrates a side view of another bipolar electrode arrangement variation where hood 12 may be internally segmented into two or more separated chambers where saline fluid having opposite charges may be introduced into each respective chamber for bipolar ablation. In the variation shown, hood 12 may have first chamber 92 and second chamber 94 divided by septum 90, which may be fabricated from the same material as hood 12 or any other number of electrically non-conductive transparent medical-grade materials, e.g., ChronoFlex™, such that both chambers and the underlying tissue may be visualized via imaging element 34. Each chamber 92, 94 may define a corresponding first and second aperture 96, 98 over the distal membrane and may also each have a corresponding first and second electrode 100, 104 positioned within each respective chamber 92, 94. Each electrode may be positioned within the chambers via respective first and second electrode support members 102, 106. The transparent fluid may be introduced into each chamber 92, 94 past the electrodes 100, 104 such that the charged fluid 108, 110 passing through their respective apertures 96, 98 may contact one another over the tissue to conduct energy 112 therebetween and ablate the underlying tissue.

In another variation, FIG. 11 shows a side view of a hood 12 having a second smaller inner hood 124 positioned within the interior of hood 12. The conductive fluid 120 may be infused into the interior of hood 12 past one or more conductive electrodes 100, 104 and over inner hood 124 and distally through die hood aperture. Fluid may be infused through an inner fluid lumen 122 containing a return electrode such that the fluid 126 infused through lumen 122 and into inner hood 124 may contact the charged fluid introduced into hood. 12. When the fluid infused through hood 12 and through lumen 122 come into contact, the underlying tissue may be ablated by the energy conducted through the fluid. In other variations, additional inner hood structures may also be contained by the outer hood for bipolar electrode ablation.

FIG. 12 shows a side view of another variation where hood 12 is replaced by an expandable balloon 130 which may be inflated by the transparent fluid. The balloon 130 may be fabricated from a transparent material to allow for visualization of contacted tissue underlying the balloon 130 by an imaging element 132 positioned within the balloon 130. Additionally, a balloon support member 134 may extend through the balloon from deployment catheter 16 to a distal end of the balloon 130 to provide structural integrity. The balloon 130 may itself be divided into two or more separate chambers each defining a respective first and second aperture 136, 138 near a distal end of balloon 130. Within each separate chamber, a first and second electrode 100, 104 may be positioned such that the conductive fluid 140, 142 flowing past each respective electrode may conduct ablation energy 144 between the electrodes 100, 104 via the conductive fluid when flowed out of the respective apertures 136, 138 and into contact with the underlying tissue T. By inflating the balloon 130 when it reaches the site of ablation, imaging element 132 can be deployed from a work channel defined in support member 134 to visualize the ablated site throughout the ablation procedure.

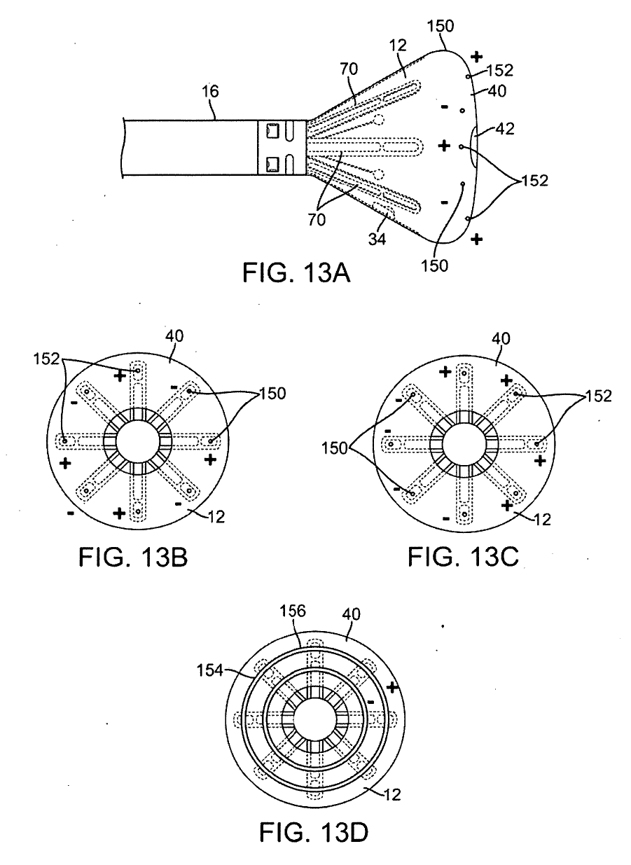

FIG. 13A shows a side view of yet another variation where the distal membrane 40 of hood 12 may have multiple conducting electrodes 150 and return electrodes 152 directly upon the face of membrane 40 for contact against the underlying tissue. The shape and size of the formed lesions can be controlled by the arrangement of bipolar electrodes across the hood membrane 40. In one arrangement shown in the end view of FIG. 13B, oppositely charged electrodes 150, 152 may be placed adjacent to each other in an alternating circumferential pattern over membrane 40. In another variation shown in the end view of FIG. 13C, similarly charged electrodes 150, 152 may be grouped together in a circumferential pattern over membrane 40. Such an arrangement may result in the formation of linear lesions which is normally desirable in ablation procedures. Another bipolar electrode arrangement is shown in the end view of FIG. 13D, which shows a conducting electrode ring 154 and a return electrode ring 156 arranged in a concentric pattern with respect to the aperture over the face of membrane 40. The flow of current passing between these oppositely charged rings 154, 156 may aid in the formation of lesions in the tissue region within the periphery of these ring electrodes. The flow rate of the saline can be regulated from the proximal end of the catheter system if so desired.

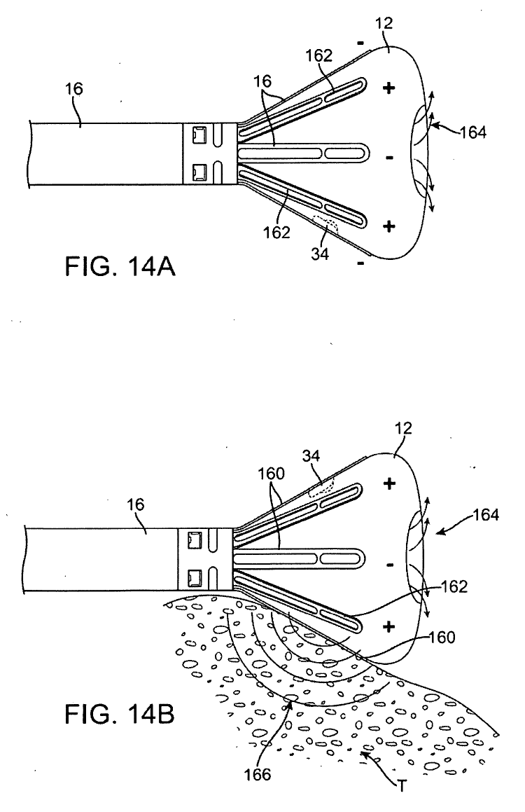

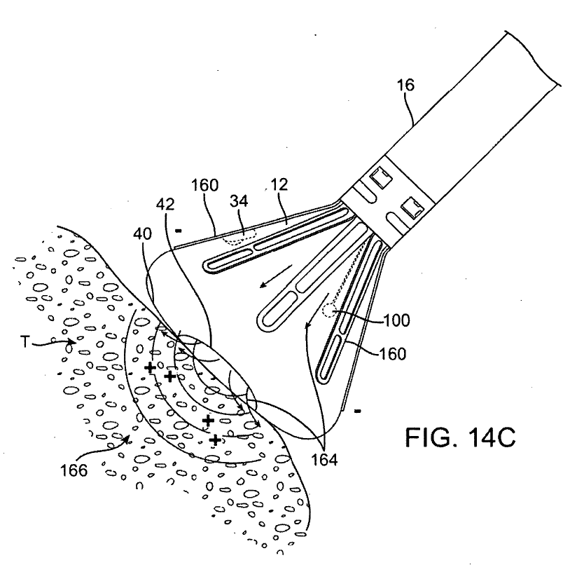

FIG. 14A shows a side view of another variation where one or more electrically conductive support struts 160 may be function as an electrode to conduct electricity to one or more corresponding return electrode support strut 162. These electrode support struts 160, 162 may be positioned along hood 12 such that they are exposed exteriorly along an outer surface of hood 12. The conductive fluid 164 flowing through hood 12 may flow out of the aperture and around the electrode struts such that energy is conducted between the struts 160, 162. Because of the positioning of the struts along an exterior surface of hood 12, the hood outer surface may be utilized to contact and ablate underlying tissue, as illustrated in the side view of FIG. 14B. The flow of ablation energy 166 through the electrically charged fluid 164 between the struts 160, 162 may result in the formation of lesions on the tissue region under the base of the hood 12 as well as along the side surfaces of the hood 12.

In yet another variation shown in the side view of FIG. 14C, an electrode 100 may be positioned within hood 12 such that the flow of conducting fluid 164 past the electrode 100 may conduct ablation energy when contacted against one or more support struts 160 configured as a return electrode to ablate the underlying tissue T. The conducting saline fluid 164 may not only purge the hood 12 of blood to facilitate visualization of the underlying tissue but may also be used to potentially cool the ablation area and ensure the formation of uniform lesions on the tissue regions T.

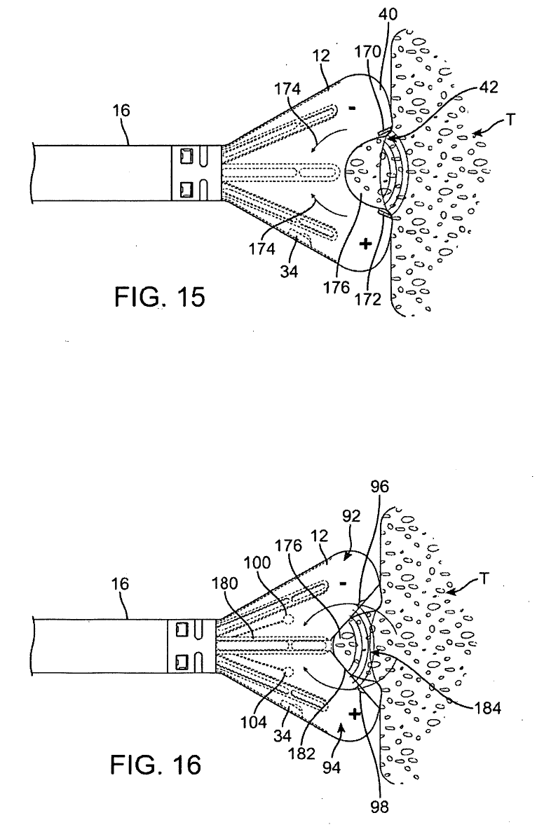

FIG. 15 illustrates a side view of another variation where a portion of tissue 176 under visualization through hood 12 may be drawn at least partially into the interior of hood 12 through aperture 42 by back-flowing the transparent fluid 174 back through a fluid lumen in deployment catheter 16. At least one conducting electrode 170 and at least one return electrode 172 may be positioned about the hood aperture 42 such that the tissue 176 pulled into the hood 12 may be subjected to ablation energy conducted between the two electrodes 170, 172 while under direct visualization from imaging element 34. Such an electrode arrangement may enable lesion formation across the entire depth of tissue 176 in a more efficient and predictable manner as compared to surface ablation.

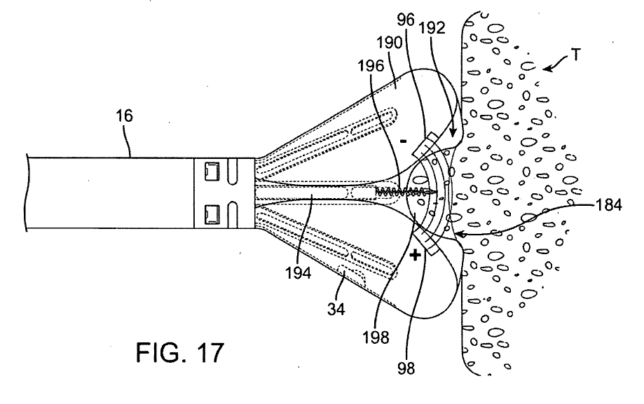

FIG. 16 illustrates another variation also utilizing suction where hood 12 may be segmented into at least two chambers where each chamber defines a respective aperture 96, 98, as previously described. The targeted tissue T may be adhered via back-flowed saline or via a separate suction lumen 180 to pull a portion of tissue 176 into a working space or theater 182 defined between the apertures 96, 98. Ablation energy 184 may be subsequently conducted through the saline fluid between the respective electrodes 100, 104 positioned within each chamber such that the adhered tissue 176 is ablated through its thickness as well as the underlying tissue T.

FIG. 17 shows a side view of yet another variation where hood 12 may be replaced by a toroidal balloon 190 which may be fabricated from a transparent material, as previously described. Toroidal balloon 190 may be tapered to extend radially away from catheter 16 while defining a working space or theater 192 within the balloon interior. A tissue grasper 196, e.g., helical grasper, extending from an instrument shaft 194 may be translatable through catheter 16 and working space 192 such that tissue to be ablated may be engaged by the grasper 196 and pulled proximally into working space 192. The grasped tissue 198 may be brought into contact against electrodes 96,98 positioned about the working space 192 along balloon 190 such that ablation may be effected upon the tissue.

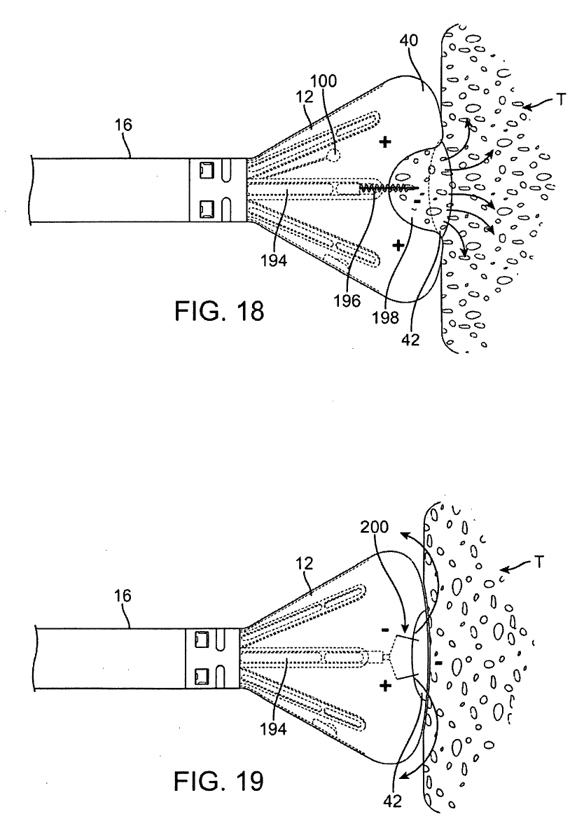

Another variation is shown in the side view of FIG. 18, which shows a tissue grasper 196 engaging and pulling a portion of tissue 198 to be ablated at least partially into the hood 12 through aperture 42. In this variation, grasper 196 may be configured as an electrode such that ablation energy may be conducted between the grasper 196 and electrode 100 via the saline fluid and through the grasped tissue 198.

FIG. 19 shows another variation in the side view of an imaging hood 12 having an instrument shaft 194 with a tissue grasper 200 having at least two members configured for engaging tissue and also for functioning as electrodes in a bipolar arrangement. The mechanical action of the grasper 200 enables the engagement of a tissue fold through which ablation energy may be conducted. The members of grasper 200 can be manipulated by applying a push or pull force at the proximal end of the catheter.

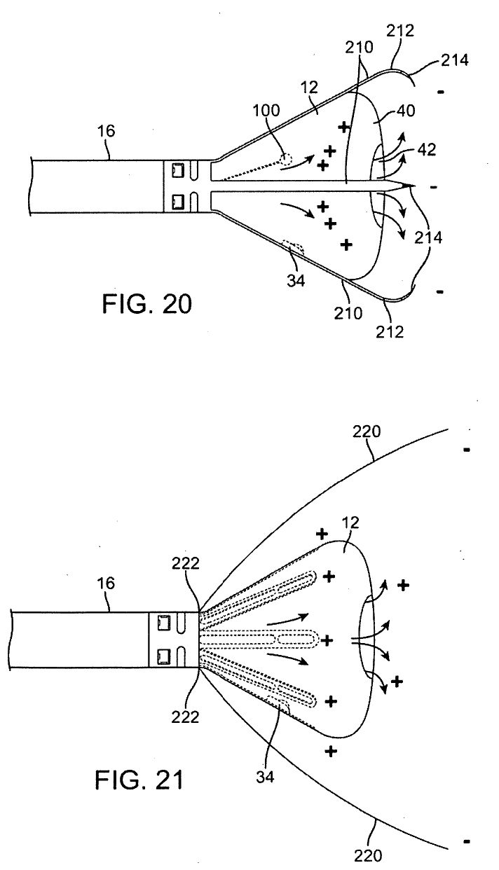

FIG. 20 shows yet another variation in a side view of an assembly which may be used to controllably form lesions which are relatively wider than an area of the hood distal membrane 40. One or more electrically conductive struts 210 may have a projecting portion 212 extending distally at an angle from hood 12 and terminating at a conducting tip 214 which may be optionally tapered into a needle-like tissue piercing tip. In use, as the underlying tissue is visualized, as previously described, the one or more conducting tips 214 may be extended distally into the tissue region surrounding the hood 12 contacted against the tissue surface and the conducting fluid may be infused into hood 12 past electrode 100 and through aperture 42 and into the area immediately surrounding hood 12. The ablation energy may be thus conducted between electrode 100 and the one or more conducting tips 214 via the fluid to ablate the tissue therebetween. The conducting tips 214 extended by the projecting portions 212 may thus result in effective and relatively deeper transmural ablation of the tissue area not only directly beneath hood 12 but also the tissue surrounding the hood 12.

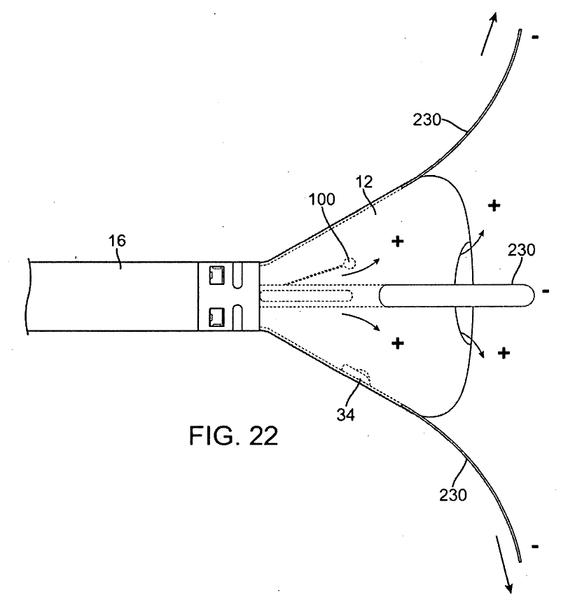

FIG. 21 shows a side view of an alternative variation where one or more conductive wires 220 may extend distally past hood 12 from attachment points 222 located proximal to hood 12. In a similar manner, the one or more conductive wires 220 may extend linearly or in an arcuate manner distal to hood 12 such that when hood 12 is positioned against a tissue region to be treated, the conductive wires 220 contact the tissue region surrounding the hood 12 to create a lesion pattern extending beyond the hood 12. FIG. 22 shows yet another alternative utilizing one or more distally extendable conducting struts 230 which may be extended from hood 12 such that the struts 230 curve and extend radially from hood 12 within a plane formed by the distal membrane of hood 12. The extendable struts 230 may function as return electrodes for the ablation energy conducted from electrode 100 within hood 12 via the conductive transparent fluid flowing through the aperture. Depending upon the size of desired lesion, the distance which the struts 230 extend from hood 12 can be controlled.

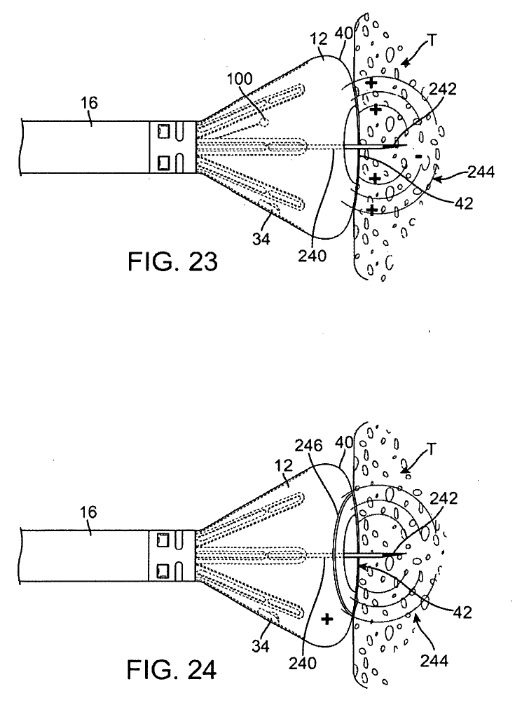

FIG. 23 depicts a side view of another variation where a transmural needle tip advanceable through hood 12 and into underlying tissue T may be used as a return electrode to create relatively deeper lesions. Needle support member 240 may comprise a piercing conductive needle tip 242 which may be advanced through aperture 42 and into the underlying visualized tissue. With electrode needle tip 242 inserted at a predetermined depth into the tissue T, ablation energy 244 may be conducted through the saline between electrode 100 and needle tip 242 to ablate the tissue T transmurally. FIG. 24 shows an alternative variation where an electrode ring 246 may be positioned along the membrane 40 in contact against the tissue surface such that the ablation energy 244 is conducted between electrode ring 246 and needle tip 242 positioned within the tissue T. The conductive saline fluid introduced into the hood 12 may thus serve in aiding in visualization of the tissue, optionally cooling of the tissue region being ablated, and may also serve as a transparent electrode for bipolar electrode ablation between the tissue in contact with the saline and the transmural needle tip 242 penetrated subsurface to the ablation site.

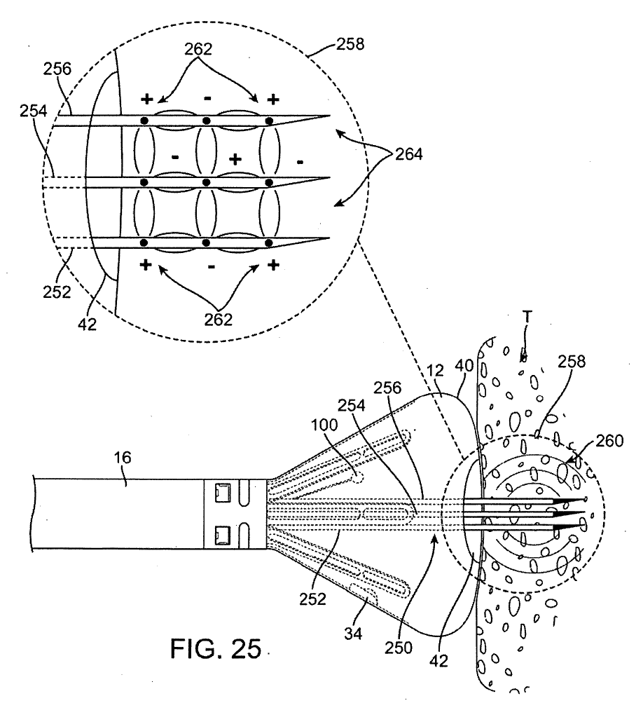

FIG. 25 shows yet another alternative variation where ablation needle assembly 250 having a number of needles 252, 254, 256 (e.g., three in this example although two needles or four or more may also be utilized, as practicable) may be advanced through hood 12 and into the underlying tissue T while positioned adjacent to one another. Each of the needles may have a distal portion, as illustrated in ablation tip detail 258, which comprises a plurality of exposed electrodes 262 at or proximal to the needle tips 264. As the needle assembly 250 is introduced into the tissue while under visualization via imaging element 34, as previously described, energy may be conducted between the exposed electrodes 262 along needle assembly 250 to ablate 260 the tissue T surrounding the needles. The flow of current between the electrodes 262 can result in isolated resistive heating at the specific subsurface tissue region. Further details of transmural needles which may be utilized with apparatus and methods described herein are described in detail in U.S. Pat. Apps. 11/828,267 filed Jul. 25, 2007 (U.S. Pat. Pub. No. 2008/0033290 A1) and 11/775,837 filed Jul. 10, 2007 (U.S. Pat. Pub. No. 2008/0009747 A1), each of which has been incorporated herein by reference above.

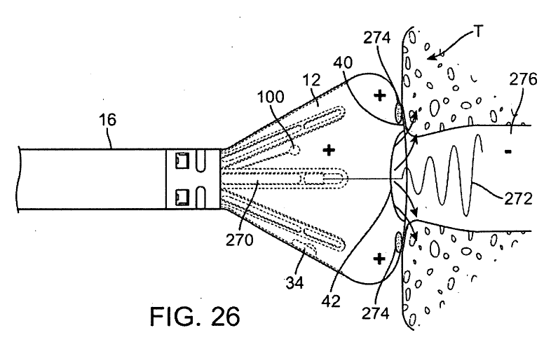

FIG. 26 shows yet another example in the side view illustrating hood 12 utilized with an anchoring member 272, e.g., a helical anchor, extendable via an instrument support member 270 which may also be configured as an electrode. For example, anchoring member 272 may be comprised of a wire member made of gold or silver coated Nitinol. Examples of helical anchoring members and methods of use are described in further detail in U.S. Pat. App. 11/959,158 filed Dec. 18, 2007 and U.S. Prov. Pat. App. 60/870,598 filed Dec. 18, 2006, each of which is incorporated herein by reference in its entirety. In this example, anchoring member 272 may be advanced in a low-profile into a vessel lumen 276, such as a lumen of a pulmonary vein, and expanded to temporarily engage the vessel walls. The circumference of membrane 40 in contact against the lumen ostium may comprise one or more return electrodes 274 such that when the electrodes are energized, current may flow between the electrodes via the saline fluid flowing past to ablate the surrounding tissue region T while under direct visualization, e.g., via imaging element 34.

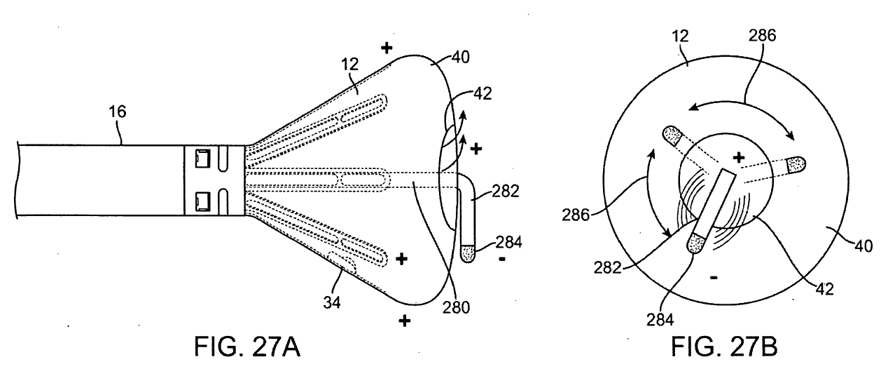

FIGS. 27A and 27B show side and end views, respectively, of another variation where current may flow between an ablation instrument extending through hood 12 having an electrode support member 280 with an angled portion 282 and at least one conducting electrode 284 positioned near or at a distal end thereof. Electrode 284 may be rotatable about a longitudinal axis of support member 280, as indicated by the direction of rotation 286, to facilitate placement of the electrode 284 over the underlying visualized tissue for optimizing ablation. One or more support struts along hood 12 may be configured as an electrode such that the ablation current may be conducted through the saline fluid between electrode 284 and the one or more electrodes of the support struts.

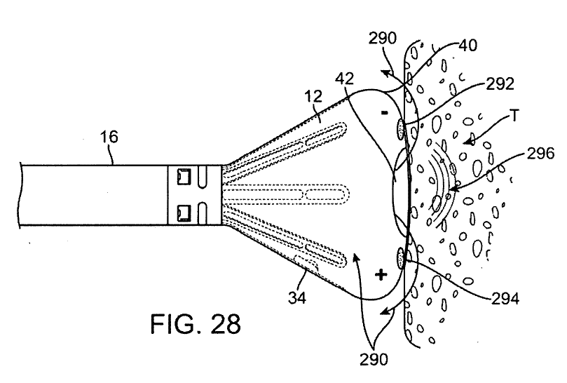

FIG. 28 shows a side view of yet another variation where hood 12 may be configured with electrode 292, 294 arranged over the membrane 40, as previously described. In this variation, or in any of the other configurations described herein, the salinity and/or temperature of the saline fluid may be controlled or altered to affect the conductivity of the saline fluid. For example, a transparent fluid 290 having its salinity altered (e.g., hyposaline fluid having a salt concentration less than 0.9%) may be introduced through hood 12 to reduce the conductivity of the fluid 290 and to accordingly adjust the ablation energy 296 through the tissue T. Additionally and/or alternatively, a temperature of the fluid 290 may also be altered to further control a conductivity of the fluid (e.g., saline fluid having a temperature of 20° C.). Moreover, the use of a hyposaline fluid 290 (and/or optionally reduced in temperature relative to body temperature) may reduce the conductivity of the surface of the ablated tissue region to potentially increase the depth of the ablated lesion as higher power and/or longer ablation durations can be applied without charring, desiccating, or causing endothelial disruption to the tissue surface. This can be applied with both monopolar and bipolar electrode arrangements.

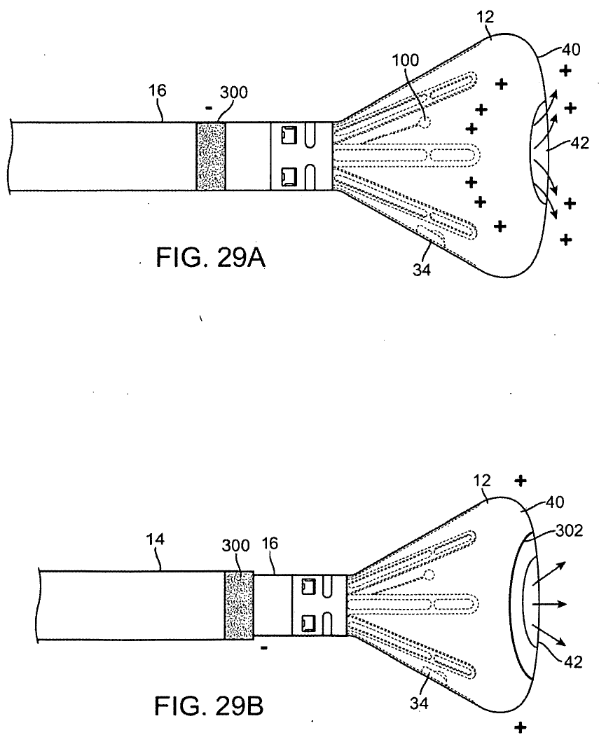

FIGS. 29A and 29B show side views of additional variations where a return electrode may be positioned externally and proximally to hood 12. In the example of FIG. 29A, return electrode 300 may be positioned proximal to hood 12 along a portion of the deployment catheter 16 while in the example of FIG. 29B, return electrode 300 may be positioned proximal to hood 12 along the outer sheath 14, in which case a position of the electrode 300 may be adjusted by movement of the sheath 14 and/or catheter 16 relative to one another. The example in FIG. 29B also illustrates the use of an electrode ring 302 positioned about aperture 42 over membrane 40, although other electrode variations may be utilized.

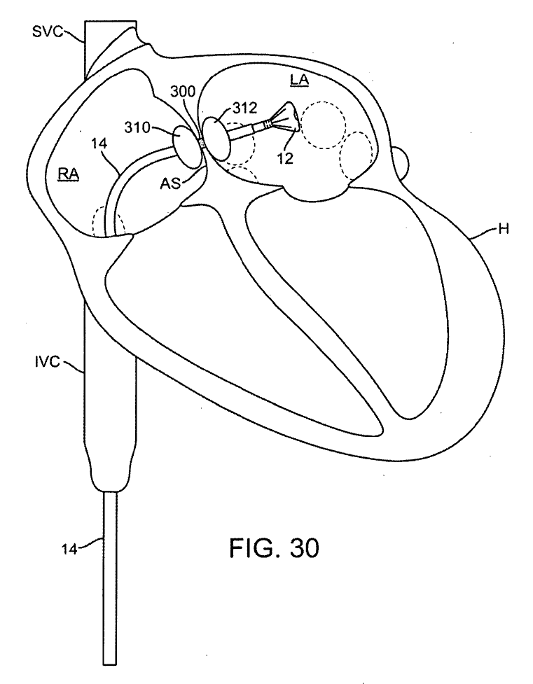

FIG. 30 illustrates one example of use in a patient heart H where the electrode 300, positioned in this example along outer sheath 14, may be positioned adjacent or proximate to the atrial septum AS with the hood 12 extending through the atrial septum AS and into the left atrium LA. The device may be advanced intravascularly, e.g., through the inferior vena cava IVC and the right atrium RA. The superior vena cava SVC is also illustrated for reference. A position of the electrode 300 may be optionally maintained relative to the atrial septum AS via one or more stabilizing balloons 310, 312 inflated on one or both sides of the septum. Electrode 300 may thus serve as a return electrode for ablation via an electrode positioned within or along hood 12, as previously described.

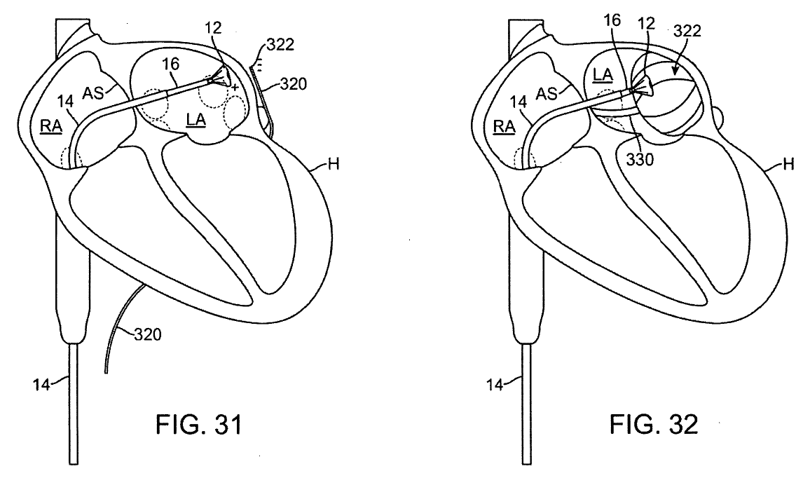

In the example shown in FIG. 31, hood 12 may be advanced within the patient heart H as previously described, yet an electrode catheter 320 having a return electrode 322 positioned therealong may be advanced as an instrument separate from the visualization assembly and positioned in proximity to the hood 12. In this example, return electrode 322 may be placed along an external surface of the patient heart H, e.g., along the epicardial wall via a thoracotomy or through a vessel within the heart in proximity to the ablation site, such as through the coronary sinus, pulmonary artery, superior vena cava, inferior vena cava, etc.

FIG. 32 shows another example where the hood 12 may be positioned, e.g., within the left atrium LA as previously described but with a return electrode 332 positioned intravascularly via a separate electrode catheter 330 such that the return electrode 332 is positioned endocardially. The return electrode 332 may take the shape, in one example, of a shape memory expandable basket with a plurality of electrodes forming the frame of the basket, as shown. This electrode basket can be made from a conductive material such as gold or silver-coated Nitinol.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue treatment apparatus, comprising:
a reconfigurable structure extending from a deployment catheter and which is capable of intravascular delivery in a low profile delivery configuration and expansion to a deployed configuration which defines an open area bounded at least in part by the structure;
a fluid lumen in communication with the open area of the structure such that introduction of a conductive fluid through the lumen purges the open area of blood when the structure is further bounded by a tissue surface;
a first electrode positioned within or along the structure and at least one second electrode also within or along the structure in proximity to the first electrode, whereby current is conducted between the first and second electrodes such that tissue between the first and second electrodes is treated.

2. The apparatus of claim 1 further comprising an imaging element within or along the structure such that the open area is contained within a visual field of the imaging element.

3. The apparatus of claim 1 wherein the first electrode is positioned within the structure such that the conductive fluid flows past the first electrode.

4. The apparatus of claim 1 wherein the first electrode comprises a ring electrode positioned upon a distal membrane of the structure in contact against the tissue surface.

5. The apparatus of claim 1 wherein the first and second electrodes comprise a plurality of electrodes positioned in a circumferential arrangement over a distal membrane of the structure.

6. The apparatus of claim 1 wherein the first electrode and second electrode each comprise a support strut positioned along the structure.

7. The apparatus of claim 1 wherein the at leas tone second electrode comprises a ring electrode positioned upon a distal membrane of the structure.

8. The apparatus of claim 1 wherein the at least one second electrode is positioned upon a distal membrane of the structure.

9. The apparatus of claim 1 wherein the structure is divided into at least two chambers such that the first electrode is positioned within or along a first chamber and the second electrode is positioned within or along a second chamber, wherein the first chamber and second chamber are in fluid communication with one another.

10. A method of treating tissue, comprising:
intravascularly advancing a reconfigurable structure in a low profile delivery configuration extending from a deployment catheter;
reconfiguring the structure to a deployed configuration which defines an open area bounded at least in part by the structure;
positioning the structure against a tissue surface to be treated such that the open area is further bounded at least in part by the tissue surface;
purging the open area of blood with a conductive fluid; and
conducting a current via the conductive fluid between a first electrode positioned within or along the structure to at least one second electrode also within or along the structure in proximity to the first electrode such that tissue between the first and second electrodes is treated.

11. The method of claim 10 further comprising visualizing the tissue surface via an imaging element within or along the structure while conducting the current.

12. The method of claim 10 wherein conducting a current comprises conducting the current between the first electrode positioned within the structure and the second electrode positioned along an outer surface of the structure.

13. The method of claim 10 wherein conducting a current comprises conducting the current between the first electrode and the second electrode configured as an instrument distally extendable past the structure.

14. The method of claim 13 wherein the instrument comprises a tissue grasper.

15. The method of claim 13 wherein the instrument comprises an expandable anchor.

16. The method of claim 13 wherein the instrument comprises one or more piercing needles.

17. The method of claim 13 wherein the instrument comprises a conductive wire distally extendable past the structure.

18. A tissue treatment apparatus, comprising:
a reconfigurable structure extending from a deployment catheter and which is capable of intravascular delivery in a low profile delivery configuration and expansion to a deployed configuration which defines an open area bounded at least in part by the structure;
a fluid lumen in communication with the open area of the structure such that introduction of a conductive fluid through the lumen purges the open area of blood when the structure is further bounded by a tissue surface;
a first electrode positioned in proximity to the structure and at least one second electrode positioned in proximity to the first electrode, whereby current is conducted between the first and second electrodes such that tissue between the first and second electrodes is treated.

19. The apparatus of claim 18 further comprising an imaging element within or along the structure such that the open area is contained within a visual field of the imaging element.

20. The apparatus of claim 18 wherein the first electrode comprises an instrument advanceable through the structure.

21. The apparatus of claim 20 wherein the instrument comprises a tissue grasper.

22. The apparatus of claim 20 wherein the instrument comprises one or more piercing needles.

23. The apparatus of claim 18 wherein the first electrode comprises a ring electrode positioned upon a distal membrane of the structure in contact against the tissue surface.

24. The apparatus of claim 18 wherein the first electrode comprise a support strut positioned along the structure.

25. The apparatus of claim 18 wherein the at least one second electrode comprises a conductive wire distally extendable past the structure.
